(19) United States
(12) Patent Application Publication
(10) Pub. No.: US 2019/0083022 A1
(43) Pub. Date: Mar. 21, 2019

(54) FATIGUE DRIVING MONITORING DEVICE

(71) Applicant: Hsin Ming Huang, Kaohsiung City (TW)

(72) Inventor: Hsin Ming Huang, Kaohsiung City (TW)

(21) Appl. No.: 15/834,872

(22) Filed: Dec. 7, 2017

(30) Foreign Application Priority Data
Sep. 15, 2017 (TW) 106213767

(51) Int. Cl. A61B 5/18; G08B 21/06; A61B 5/00; A61B 5/024; G01S 5/02; B60W 10/18; B60W 30/09; B60W 50/14

(52) U.S. Cl. CPC A61B 5/18; G08B 21/06; A61B 5/6824; A61B 5/024; B60W 2040/0827; B60W 10/18; B60W 30/09; B60W 50/14; G01S 5/0284

(57) ABSTRACT

A fatigue driving monitoring device includes a detector wearable on any part of a driver's body and a receiver disposed in the body of a vehicle or mounted in the body of the vehicle in a plug-in manner. The receiver is connected to the detector. The detector serves to detect the physiological data of the driver, such as heart rate, blood oxygen, heart rate variation and blood pressure. The physiological data are transmitted to the receiver via a transmission system and then the main control motherboard of the detector calculates, evaluates, analyzes and judges the current fatigue state of the driver. In case the detector detects that the driver is in a high-fatigue state or reaches a warning value, the receiver immediately actively emits a warning signal to remind the driver and a message is also transmitted to a control center to perform proper action.

### BACKGROUND OF THE INVENTION

### 1. Field of the Invention

The present invention relates generally to a fatigue driving monitoring device, and more particularly to a monitoring device, which has higher fatigue detection effect.

### 2. Description of the Related Art

The number of various vehicles has been more and more increased. As a result, the possibility of traffic accidents has become higher and higher. The major causes of the traffic accidents are nothing else but drunk driving, over-speeding and physiological factors. With respect to the physiological factors, most of the physiological problems take place suddenly, unexpectedly and unavoidably. For example, fatigue driving will seriously affect the traffic safety. The danger caused by fatigue driving is much higher than other causes of traffic accidents.

In the recent years, along with the development of intelligent transportation system, the detection technique that employs various advanced sciences and technologies to detect the mental condition of a driver or avoid driving negligence has been quite widely applied. The conventional detection technique can be generally classified into several manners as follows:
1. In the case the speed per hour of the vehicle exceeds 65 km/h and such situation lasts for over 15 minutes, the fatigue warning system will continuously operate to detect the driving conditions of the driver, (such as the rotation of the steering wheel and abnormal deflection or acceleration), so as to judge whether the driver is in a fatigue state. In case a fatigue state is detected, the dashboard will show a signal (such as a coffee cup) to suggest the driver to take a rest and emit a warning sound to remind the driver.
2. The driver attention assistant system operates in such a manner that a sensor is disposed in the steering wheel to detect the hand action of the driver operating the steering wheel and analyze whether the driver has abnormally steered the steering wheel. In addition, the display screen on the dashboard will show a mark of a coffee cup to remind the driver to pay attention to safety.
3. A set of infrared sensors are mounted on the dashboard. The infrared sensors will continuously emit infrared rays to detect the eyeballs motion of the driver. In case it is found that the driver's eyelids have been closed or the driver's eyes are staring in a direction other than the drive lanes, the computer will remind the driver by means of such as emitting a warning sound to wake up the driver.
4. In the case that a driver deflects from the driving lane too many times, the lane deflection warning system will judge that the driver is in the fatigue driving state. At this time, the display screen on the dashboard will show a mark of a coffee cup to remind the driver to stop driving the vehicle and take a rest.
5. The driver lethargic sleep detection system employs a turning angle sensor or electrical turning to continuously analyze the turning behavior of the driver so as to find the standard fatigue form. The system will also record any sudden and minor turning motion. With the assistance of other parameters (such as the driving hours and the time of the same time), the system can find the fatigue sign and give the warning.

The above means can detect the physiological motion of a driver. However, the detection effect is varied with the difference between the physiological conditions of the individuals. Therefore, it is hard to make a precise and optimal judgment and the detection effect is limited.

### SUMMARY OF THE INVENTION

It is therefore a primary object of the present invention to provide a fatigue driving monitoring device, which includes a detector and a receiver connected to the detector. The wearable transceivers connected to the detector are used to monitor the physiological data such as the blood oxygen, heart rate, heart rate variation (HRV), breathing frequency and blood pressure of the driver. Via a transmission system (such as Bluetooth technique), the data are transmitted to the receiver in the form of an onboard apparatus or a personal plug-in apparatus. The main control motherboard of the detector calculates to evaluate the fatigue index of the driver so as to analyze and judge the current fatigue state of the driver. In case the detector detects that the driver is in a high-fatigue state or reaches a fatigue warning value, the detector and the receiver will immediately actively emits a warning signal to remind the driver and a message is also transmitted to a control center to perform proper action.

To achieve the above and other objects, the fatigue driving monitoring device of the present invention includes a detector and a receiver. The detector includes a watch case with a display screen and a main control motherboard disposed in the watch case. One or more transceivers are disposed on the watch case and connected to the main control motherboard. The transceivers can emit light beams. The detector is wearable on any part of a driver's body to attach to and contact the part. The transceivers connected to the main control motherboard are photoplethymography (PPG) transceivers, which mainly employ photosensors to absorb the optical energy so as to record the change of light and generate signals. That is, during the heart diastole and systole period, the blood flow amount per unit area in the vessel will periodically change. When the blood volume varies, the photosensors will sense the voltage with the change of the blood volume so as to change the output potential. The change of the output potential is processed by a posterior circuit to indicate the heart rate change. Finally, the detected physiological parameters are converted into wireless transmission form and the data are transmitted out in a wireless manner. On the basis of the variation period of the signal and the ratio of the minimum and maximum detector signals, the physiological data such as the blood oxygen, heart rate, heart rate variation, breathing frequency and blood pressure of the driver can be analyzed and measured. The receiver has the form of an onboard (BLE gateway) apparatus or a personal plug-in apparatus. The receiver is connected to the detector for receiving the detection signal of the detector. A calculation circuit board of the main control motherboard of the detector serves to evaluate, analyze and judge the current fatigue state and extent of the driver and transmit the fatigue state and extent of the driver to the receiver.

Accordingly, after the vehicle is started, the detector of the present invention can automatically mate and connect with the onboard (BLE gateway) apparatus or the personal plug-in apparatus of the receiver. The detector and the receivers are immediately activated to operate. The detector serves to monitor the physiological behaviors of the driver and the transceivers connected to the detector serve to collect the physiological data such as the blood oxygen, heart rate, heart rate variation, breathing frequency and blood pressure of the driver. The main control motherboard of the detector serves to calculate and evaluate the fatigue index HRV<50 ms² (50 ms² is the threshold in millisecond literature) so as to analyze and judge the current fatigue state of the driver. When the fatigue data appear, the receiver immediately activates a warning signal to remind the driver and the detector simultaneously remind the driver via vibration. Also, a message is also immediately transmitted to a third-party security system, which immediately activates the monitoring.

In case the warning signal exceeds 5 to 10 seconds and the physiological data such as the heart rate, blood oxygen, blood pressure and heart rate variation the driver still fail to restore to the standard value or the driver fails to immediately properly react and adjust his physical and mental conditions, the third-party security system (a security company or a service center) immediately calls to the receiver to inform the driver with the phone (the phone device is set to forced automatic answering) so as to remind and avoid the behavior of fatigue driving.

The present invention can be best understood through the following description and accompanying drawings, wherein:

### BRIEF DESCRIPTION OF THE DRAWINGS

FIG. 1 is a perspective view of the detector of the fatigue driving monitoring device of the present invention;

FIG. 2 is a front view of the detector of the fatigue driving monitoring device of the present invention;

FIG. 3 is a front view of the receiver of the fatigue driving monitoring device of the present invention; and

FIG. 4 is a block diagram showing the use of the fatigue driving monitoring device of the present invention.

### DETAILED DESCRIPTION OF THE PREFERRED EMBODIMENTS

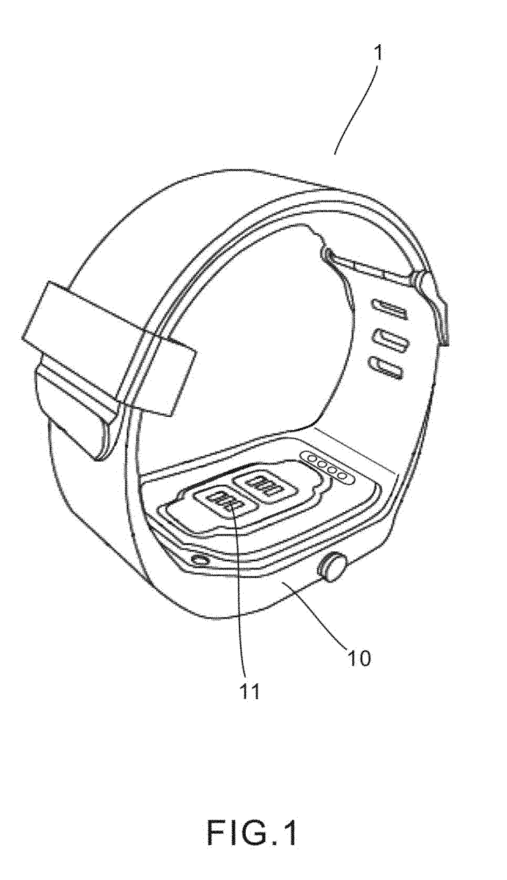

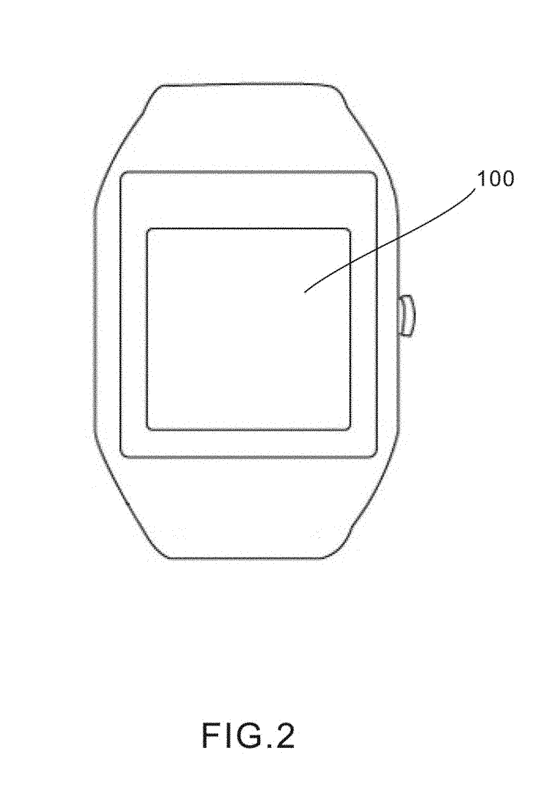

The fatigue driving monitoring device of the present invention includes a detector 1 and a receiver 2. With reference to FIGS. 1 and 2, the detector 1 can have the form of a watch including a watch case 10 with a display screen 100 and a main control motherboard (not shown) disposed in the watch case 10. One or more transceivers 11 are disposed on the back face of the watch case 10 and electrically connected to the main control motherboard. The transceivers 11 can respectively emit red light or green light. The watch case 10 is attached to and in contact with the wrist of a driver, whereby the transceivers 11 employ photosensors to absorb optical energy so as to record the change of light and generate signals. That is, during the heart diastole and systole period, the blood flow amount per unit area in the vessel will periodically change. When the blood volume varies, the photosensors will sense the voltage with the change of the blood volume so as to change the output potential. The change of the output potential is processed by a posterior circuit to indicate the heart rate change. Finally, the detected physiological parameters are converted into wireless transmission form and the data are transmitted out in a wireless manner. On the basis of the variation period of the signal and the ratio of the minimum and maximum detector signals, the physiological data such as the blood oxygen, heart rate, heart rate variation, blood pressure and breathing frequency of the driver can be collected and analyzed. Moreover, by means of a transmission system (such as Bluetooth transmission or Wi-Fi), the collected physiological data are transmitted to the receiver. The detector 1 can have some other forms such as a bracelet, an earphone, a finger ring, an arm strap, a pasties, a chest strap, a pair of glasses, a leg ring or any other portable equipment that can detect the physiological data.

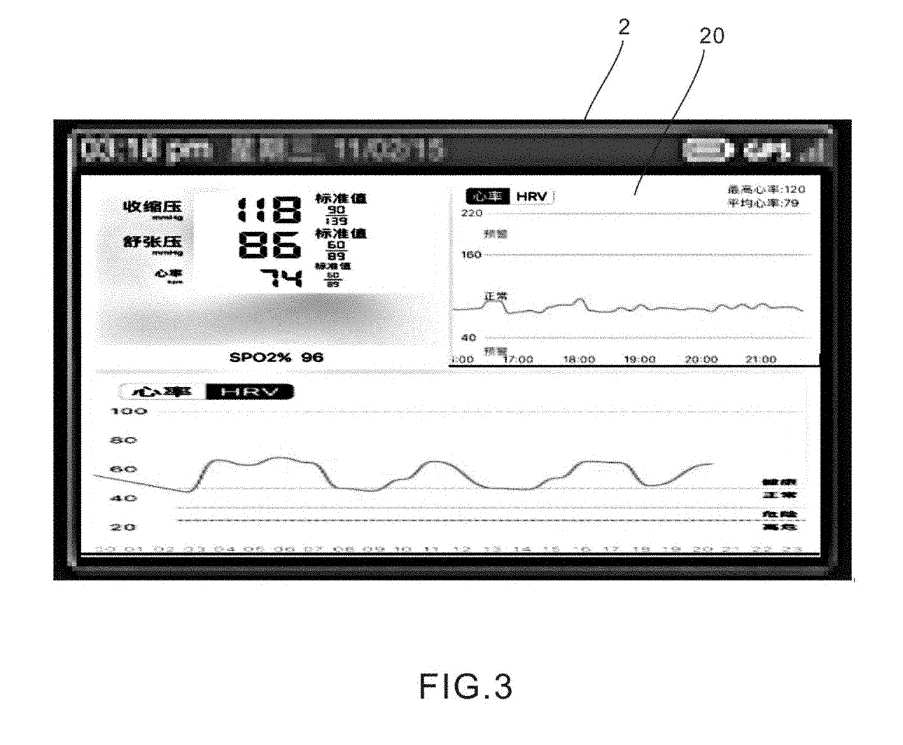

As shown in FIG. 3, the receiver 2 is disposed in the vehicle in the form of an onboard apparatus (such as an inbuilt driving computer or an external computer) or a personal plug-in apparatus. The receiver 2 is connected to the detector for receiving the physiological data transmitted from the detector. The assembled equipments of the receiver mainly includes a display 20, a high-quality speaker and microphone, a main control motherboard, a GPS satellite positioning integrated module, a wearable device communication protocol, a lithium battery, a USB and a SIM card slot. The display serves to display the physiological data such as the blood oxygen, heart rate, heart rate variation (HRV), blood pressure and breathing frequency. The speaker serves to emit a warning sound. The microphone is used to respond to the current situation. The SIM card is inserted into the SIM card slot as a Hotpoint for data transmission and calling to inform whether there is an emergency condition. The receiver 2 can have some other forms such as a wearable communication watch, a bracelet, an independent plug-in apparatus, a dashcam, a navigator, a helmet or any other plug-in onboard display or a rearview mirror.

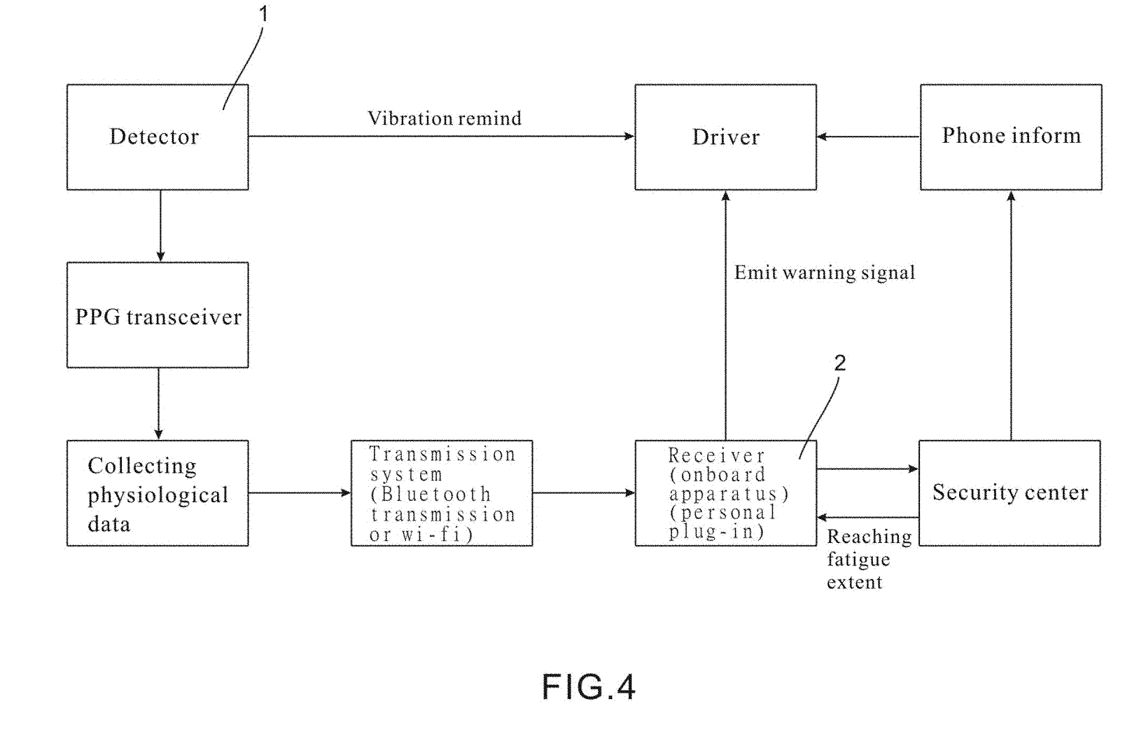

With respect to the use procedure of the present invention, as shown in FIG. 4, the detector 1 and the receiver 2 are connected via the transmission system (such as Bluetooth technique). After the vehicle is started, the transceivers 11 connected to the main control motherboard of the detector 1 directly monitor the can have some other forms such as a bracelet, an earphone, a finger ring, an arm strap, a pasties, a chest strap, a pair of glasses, a leg ring or any other portable equipment that can detect the physiology of the driver to collect the physiological data such as the blood oxygen, heart rate, heart rate variation (HRV), blood pressure and breathing frequency of the driver. Via the transmission system (such as Bluetooth technique), the data are transmitted from the detector to the receiver. The main control motherboard of the detector calculates to directly analyze and evaluate the fatigue index of the driver. The fatigue index is based on the heart rate variation (HRV). In normal state, the heart rate variation must be smaller than 50 ms. Otherwise, it is judged that the driver has reached the fatigue warning value. After the fatigue index is obtained, the fatigue index is transmitted to the receiver 2 into the monitoring mode.

When the driver is monitored and judged to be in a fatigue state, the detector 1 immediately two-way informs the receiver 2 and a third-party security system. The receiver immediately activates the warning signal to warn the driver via the speaker. The detector synchronously reminds the driver via vibration signal, while the third-party security system immediately monitors. In case the warning signal exceeds 5 to 10 seconds and the heart rate, the blood oxygen, the blood pressure and the heart rate variation (HRV) of the driver still fail to restore to the standard value or the driver fails to immediately react and adjust his physical and mental conditions, the third-party security system immediately calls to the SIM card of the receiver to force the driver to answer the phone so as to avoid the behavior of fatigue driving. In the driver still ignores the warning, the third-party security system will know the position of the driver via the GPS satellite positioning integrated module and then perform proper action.

In order to enhance the safety, the fatigue driving monitoring device of the present invention can be further connected to an automatic braking system. Therefore, according to whether the driver is in a fatigue state, the onboard computer of the vehicle can decide when the braking system should intervene in so as to enhance the safety in driving.

The above embodiments are only used to illustrate the present invention, not intended to limit the scope thereof. Many modifications of the above embodiments can be made without departing from the spirit of the present invention.

1. A fatigue driving monitoring device comprising:
a detector including a watch case with a display screen and a main control motherboard disposed in the watch case, one or more transceivers being disposed on the watch case and connected to the main control motherboard, the watch case being worn on a part of a driver's body with the transceivers attached to and in contact with the part of the driver's body, the transceivers are photoplethymography transceivers having photo sensors; and
a receiver including a display, a speaker and microphone, a SIM card slot and a receiver main control motherboard, the receiver being connected to the detector via a transmission system, the detector serving to detect the physiological data of the driver and a calculation circuit board of the main control motherboard of the detector serving to evaluate, analyze and judge a current fatigue extent of the driver, whereby in case it is detected that the driver is in a predetermined fatigue state or the fatigue extent of the driver is lower than a warning value, the receiver emits a warning signal to remind the driver and a message is also transmitted to a control center to perform a predetermined action;
wherein the detector uses the transceivers being the photoplethymography transceivers to determine the physiological data including blood oxygen, heart rate, heart rate variation, breathing frequency and blood pressure of the driver.

2. The fatigue driving monitoring device as claimed in claim 1, wherein the transceivers are wearable on any part of a driver's body that detects the physiological data.

3. The fatigue driving monitoring device as claimed in claim 1,
wherein the receiver is an onboard apparatus.

4. The fatigue driving monitoring device as claimed in claim 3, wherein the receiver is an onboard apparatus, which is an inbuilt driving computer or an externally installed driving computer.

5. The fatigue driving monitoring device as claimed in claim 1, wherein the receiver is an plug-in apparatus disposed in a predetermined position of a body of a vehicle.

6. The fatigue driving monitoring device as claimed in claim 5, wherein the receiver is an plug-in apparatus and is selected from a group consisting of a wearable communication watch, a bracelet, an independent plug-in apparatus, a dashcam, a navigator or any other plug-in onboard display, and a rearview mirror.

7. The fatigue driving monitoring device as claimed in claim 1, wherein the receiver further includes a GPS satellite positioning integrated module, a wearable device communication protocol, a lithium battery and a USB.

8. The fatigue driving monitoring device as claimed in claim 1, wherein the detector and the receiver are further connected to an automatic braking system of a vehicle.
